(19) United States
(12) Patent Application Publication
(10) Pub. No.: US 2009/0046831 A1
(43) Pub. Date: Feb. 19, 2009

(54) BENCH-TOP IMAGE-GUIDED CONFORMAL IRRADIATION SYSTEM FOR LABORATORY ANIMALS

(75) Inventors: John Wai-chiu Wong, Towson, MD (US); Todd McNutt, Severna Park, MD (US); Elwood P. Armour, Baltimore, MD (US); Eric Cassel Ford, Catonsville, MD (US)

Correspondence Address: VENABLE LLP, P.O. BOX 34385, WASHINGTON, DC, 20043-9998, US

(73) Assignee: The John Hopkins University, Baltimore, MD (US)

(21) Appl. No.: 12/281,944

(22) PCT Filed: Mar. 12, 2007
(86) PCT No.: PCT/US2007/006148

§ 371 Date: Sep. 5, 2008

Related U.S. Application Data
(60) Provisional application No. 60/781,443, filed on Mar. 10, 2006.

(51) Int. Cl. A61B 6/00

(52) U.S. Cl. 378/19

(57) ABSTRACT

An image-guided irradiation system has a support stage, a stage-positioning assembly connected to the support stage, an x-ray source attached to a support arm in which the support arm is movable relative to the support stage, a flat-panel x-ray detector disposed proximate the support stage, and at least one of a collimator or an x-ray lens selectively disposable between the x-ray source and the support stage. The x-ray source is suitable to provide an imaging beam of X-rays at a first photon energy and is suitable to provide an irradiation beam of X-rays at a second photon energy. The collimator or x-ray lens is structured and arranged to define at least a width of the irradiation beam when it is disposed between the x-ray source and the support stage while the x-ray source is in operation.

### CROSS-REFERENCE TO RELATED APPLICATION AND FEDERAL GRANT INFORMATION

This application claims priority to U.S. Provisional Application No. 60/781,443 filed Mar. 10, 2006, the entire contents of which are hereby incorporated by reference.

This invention was made with support of NCI-NIH Grant No. 1R01-CA108449. The U.S. Government has certain rights in this invention.

### BACKGROUND

1. Field of Invention

The current invention relates to x-ray systems, and more particularly to image-guided conformal irradiation x-ray systems.

2. Discussion of Related Art

There are currently x-ray systems available for a variety of purposes. Computed tomography (CT) x-ray systems can provide three-dimensional x-ray images. Most conventional CT x-ray systems build up the three-dimensional image by taking two-dimensional slices that are projected to one-dimension for several slice-positions along an axial dimension of an object or subject being imaged. Cone-beam CT imaging, on the other hand, takes a series of two-dimensional images of the object, each from a different direction and then builds up the three-dimensional images from the series of two dimensional images. Other x-ray systems are used to deliver energy to a location within an object or subject, which we will refer to as x-ray irradiation systems. The energy delivery can be for the purposes of therapy, or other interaction mechanism. X-ray imaging systems and x-ray irradiation have different criteria for desired beam properties, such as energy for penetration and energy delivery effects, and beam size for selectivity of regions irradiated. Also, the x-ray energy can be important for obtaining good contrast images, such as distinguishing soft tissue from bone.

Conventional x-ray systems are not available that provide both imaging and irradiation capabilities in a compact, high-resolution, economical and/or simple structure. In addition, convention x-ray systems do not provide both imaging and irradiation capabilities in a compact, economical and/or simple structures that are suitable and have sufficient resolution for use with small laboratory animals. There is thus a need for improved x-ray systems.

### SUMMARY

An image-guided irradiation system according to an embodiment of the current invention has a support stage, a stage-positioning assembly connected to the support stage, an x-ray source attached to a support arm in which the support arm is movable relative to the support stage, a flat-panel x-ray detector disposed proximate the support stage, and at least one of a collimator or an x-ray lens selectively disposable between the x-ray source and the support stage. The x-ray source is suitable to provide an imaging beam of X-rays at a first photon energy and is suitable to provide an irradiation beam of X-rays at a second photon energy. The collimator or x-ray lens is structured and arranged to define at least a width of the irradiation beam when it is disposed between the x-ray source and the support stage while the x-ray source is in operation.

A portable image-guided irradiation system has a portable imaging and irradiation system and a portable radiation-shielding enclosure that is suitable to substantially enclose the portable imaging and irradiation system.

A method of irradiating an object with X-rays according to an embodiment of the current invention includes illuminating an object with a first imaging beam of X-rays produced by an x-ray source from a first direction, detecting X-rays from the first imaging beam of X-rays with a flat-panel x-ray detector after the illuminating to obtain first two-dimensional x-ray image data, illuminating an object with a second imaging beam of X-rays produced by the x-ray source from a second direction, detecting X-rays from the second imaging beam of X-rays with a flat-panel x-ray detector after the illuminating to obtain second two-dimensional x-ray image data, processing the first and second two-dimensional x-ray image data to determine a three-dimensional x-ray image of the object, irradiating a selected portion of the object with an irradiation beam of X-rays produced by the x-ray source based on the three-dimensional x-ray image of the object. The irradiation beam of X-rays is narrower than the imaging beam of X-rays.

### BRIEF DESCRIPTION OF THE DRAWINGS

Additional features of this invention are described with reference to examples of some embodiments of the invention in the following with reference to the accompanying drawings, in which:

FIG. 1 is a CAD drawing in perspective view of an image-guided irradiation system according to an embodiment of the current invention.

### DETAILED DESCRIPTION

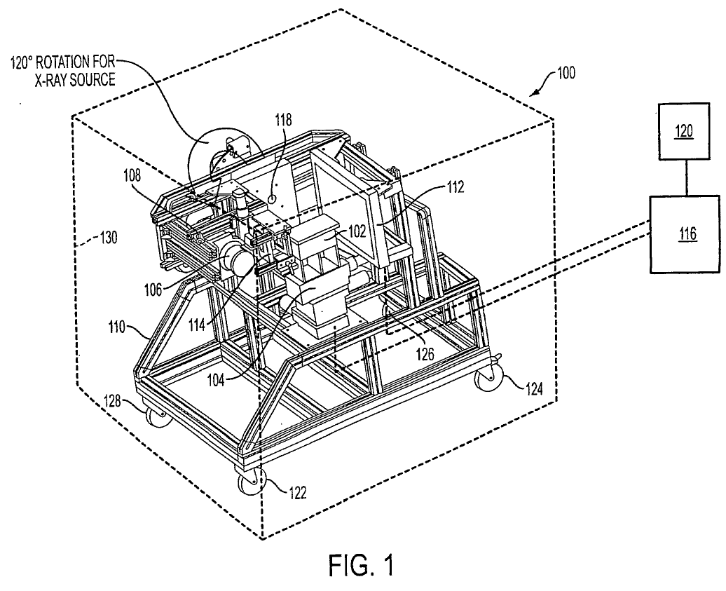

FIG. 1 is a perspective view of an image-guided irradiation system 100 according to an embodiment of the current invention. The image-guided irradiation system 100 has a support stage 102 and a stage-positioning assembly 104 connected to the support stage. An x-ray source 106 is attached to a support arm 108 that is movable with respect to the support stage 102. The support arm 108 may be pivotably connected to a support frame 110, but the general concepts of this invention are not limited to only that particular construction. A flat-panel x-ray detector 112 is disposed proximate the support stage 102. The flat-panel detector 112 may be attached to the support frame 110 in some embodiments. A collimating unit 114 is disposed between the x-ray source 106 and the support stage 102. The collimator 114 is removable so that it can be selectively disposed between the x-ray source 106 and the support stage 102. The collimating unit 114 may also be connected to the support arm 108 in some embodiments of this invention so that it can be moved along with the support arm 108 while it is moved to reposition the x-ray source 106. Alternatively, or in addition, an x-ray lens may be selectively disposed between the x-ray source 106 and the support stage 102.

The x-ray source 106 is of a type that is suitable to provide an imaging beam of X-rays at a first energy and to also provide an irradiation beam of X-rays at a second energy. The x-ray source 106 may be an x-ray tube according to an embodiment of the current invention. For example, a GE 225 x-ray tube is suitable for some applications, such as for use with small laboratory animals. However, the general concepts of the invention are not limited to only these particular examples of x-ray sources. The x-ray tube in this particular embodiment for the x-ray source 106 may be adjustable with a range of voltages to provide at least imaging X-rays as well as irradiation X-rays. In particular, the x-ray source 106 may provide a cone-beam imaging beam of X-rays for imaging an object on the support stage 102. The process of cone beam imaging is described in detail in a patent in which one of the current co-inventors is also a co-inventor (see, U.S. Pat. No. 6,842,502 issued Jan. 11, 2005 for “Cone Beam Computed Tomography with a Flat Panel Imager,” the entire contents of which are incorporated herein by reference). For the case in which the object being imaged is a small animal, such as a mouse, the voltage of the x-ray tube may be set to greater than about 70 kVp and less than about 120 kVp to obtain images of suitable contrast, for example, suitable contrast between soft tissue and bone. The cone beam produced by the x-ray source 106 can illuminate substantially an entire object on a support stage 102, for example, which is detected by the two-dimensional flat-panel detector 112.

The flat-panel detector 112 is in communication with an imaging processing system 116. The imaging processing system 116 may be a dedicated image processing system or it could be software implemented, for example, on a computer such as a personal computer. The imaging processing system 116 may be hard-wired to the flat-panel detector 112, or can have alternative communication links. The communication link between the imaging processing system 116 and the flat-panel detector 112 can include wireless communication links, for example, or a combination of hard-wired and wireless links. The image processing system 116 processes data from the flat-panel detector 112 to form a two-dimensional image of the object on the support stage 102 after it is illuminated with a cone beam of x-rays. The support arm 108 can then be moved to another position, for example, rotated about the pivot point 118, at which the object on the support stage 102 can be illuminated again with a cone-beam x-ray imaging beam to be detected by the flat-panel detector 112 in which the image processing system 116 can form a second two-dimensional projected x-ray image. This process may be repeated many times through rotations of numerous angles upon which the image processing system 116 can combine the plurality of two-dimensional images to form a three-dimensional x-ray image of the object on the support table 102. Alternatively, or in combination with this process, the x-ray source 106 can be held fixed while the object held on the support stage is rotated through many angles and/or positions. The image processing system may alternatively receive all image data at a plurality of imaging positions of the x-ray source and process such data directly to a three-dimensional x-ray image, without producing two-dimensional x-ray images, without departing from the general scope of the current invention. The image processing system 116 forms the three-dimensional x-ray image by cone-beam computed tomography in this embodiment of the current invention. Images may be displayed on a display system 120 if desired. The x-ray images may be stored, printed or handled in other suitable manners.

The stage-positioning assembly 104 may include a mechanism to move it in any one of one, two or three orthogonal linear directions, such as up and down, in and out, and back and forth in the view illustrated in FIG. 1. Furthermore, the stage may be rotatable about an axis of rotation. For example, the support stage 102 may be rotatable about an axis perpendicular to the horizontal surface upon which the object under observation may be placed (the up and down direction in FIG. 1 and sometimes conventionally referred to as the Z-direction). The stage-positioning assembly 104 is in communication with a stage control system. The stage-position control system may be a dedicated stage-position control system or can be software implemented on a multi-use computer, such as a personal computer. For example, the stage-position control system can be software implemented on the same personal computer as the image processing system 116 in the current particular embodiment. However, the broad concepts of the invention are not limited to only this particular embodiment. The stage-position control system may provide control instructions to move the support stage 102 to a position and orientation relative to the x-ray source 106 in order for an object on the support stage 102 to be irradiated by the x-ray source 106. For performing irradiation, the x-ray source 106 may be operated at a higher voltage than during imaging by the cone-beam computed tomography. For example, for the case in which the object under irradiation is a small animal, such as a mouse, one may operate an x-ray tube at greater than about 160 kVp and less than about 300 kVp to achieve suitable irradiations. In this particular application, higher energies than about 300 kVp can begin to lead to undesired damage to tissue adjacent to the treatment site and energies lower than about 160 kVp can result in insufficient depth penetration.

In addition, one may position a collimator between the x-ray source 106 and the support stage 102 to allow a sufficiently narrow beam to pass-therethrough to the desired region of irradiation. In the case of a small animal, such as a mouse, irradiation beams with widths ranging from about 0.5 mm to 60 mm can be provided with the use of collimators. This can permit one to irradiate relevant organs and regions of organs within the mouse without also irradiating undesired regions of the mouse. For example, one may irradiate a portion of the brain of the mouse with such a system. The collimator system may include one or more apertures provided in a plate of material and of a thickness to substantially block X-rays at the irradiation energy while permitting the X-rays to pass through the aperture. The collimator may be constructed from a variety of materials that are suitable for blocking X-rays. The aperture plates may be adapted so that they can be removably attached between the x-ray source 106 and the support stage 102. For example, they may be removably attachable to the support arm 108. The collimator or the aperture plates can be manually attachable and manually removable in some embodiments, or they can be moved into and out of the beam of X-rays from the x-ray source by an automated mechanism and control system.

The x-ray source may include other components such as a shutter to selectively block or allow X-rays to emanate from the x-ray source 106. One may also include attenuators and/or filters that can be placed in the beams of X-rays from the x-ray source 106 to adjust the intensity and hardness of the x-ray beams. One may also place attenuators in the beams of X-rays produced by the x-ray source 106 to improve the uniformity of the beams. In the case in which the image guided irradiation system 100 is suitable for small laboratory animals, such as laboratory mice, suitable x-ray irradiation doses can be provided by the system with a range of greater than about 50 cGy/minute and less than about 300 cGy/minute.

The image obtained through cone-beam computed tomography may be used to determine positions and orientations for an irradiation procedure. This may be done in real time and interactively in some embodiments. The three-dimensional image obtained during the cone-beam computed tomography may also be used as input to a computer program which can calculate a series of planned irradiation steps, and these irradiation steps can be automated in some embodiments of the current invention. Such a computer radiation planning can include the system parameters, for example including the x-ray source energy, x-ray beam size, x-ray beam intensity, illumination times and trajectories of the support stage.

The image guided irradiation system 100 may also include a plurality of wheels, such as wheels 122, 124, 126 and 128 attached to the support frame 110. The image-guided irradiation system 100 can be a portable image-guided irradiation system. In addition, in some embodiments of the current invention, the image-guided irradiation system 100 can be rolled from one position to another position.

The image-guided irradiation system 100 may optionally include a shielding enclosure 130 to enclose at least the x-ray source and support stage therein. The irradiation shielding enclosure 130 may substantially enclose the portable imaging and irradiation system to protect operators who may be in the same room as the image-guided irradiation system 100.

The invention has been described in detail with respect to various embodiments, and it will now be apparent from the foregoing to those skilled in the art that changes and modifications may be made without departing from the invention in its broader aspects, and the invention, therefore, as defined in the claims is intended to cover all such changes and modifications as fall within the true spirit of the invention.

We claim:

1. An image-guided irradiation system, comprising:
a support stage;
a stage-positioning assembly connected to said support stage;
an x-ray source attached to a support arm, said support arm being movable relative to said support stage;
a flat-panel x-ray detector disposed proximate said support stage; and
at least one of a collimator or an x-ray lens selectively disposable between said x-ray source and said support stage,
wherein said x-ray source is suitable to provide an imaging beam of X-rays at a first photon energy and is suitable to provide an irradiation beam of X-rays at a second photon energy; and
wherein said at least one of said collimator or x-ray lens is structured and arranged to define at least a width of said irradiation beam when it is disposed between said x-ray source and said support stage while said x-ray source is in operation.

2. An image-guided irradiation system according to claim 1, wherein said x-ray source is an x-ray tube.

3. An image-guided irradiation system according to claim 2, wherein said first photon energy corresponds to an energy in which said x-ray tube is operated at a voltage greater than about 70 kVp and less than about 120 kVp.

4. An image-guided irradiation system according to claim 2, wherein said second photon energy corresponds to an energy in which said x-ray tube is operated at a voltage greater than about 160 kVp and less than about 300 kVp.

5. An image-guided irradiation system according to claim 1, wherein said width of said irradiation beam is defined by said collimator to provide an irradiation beam of a width of about 0.5 mm at an irradiation zone proximate said support stage.

6. An image-guided irradiation system according to claim 4, wherein said width of said irradiation beam is defined by said collimator to provide an irradiation beam of a width of about 0.5 mm at an irradiation zone proximate said support stage.

7. An image-guided irradiation system according to claim 4, wherein said irradiation beam delivers a dose greater than about 50 cGy/minute and less than about 3.00 cGy/minute.

8. An image-guided irradiation system according to claim 6, wherein said irradiation beam delivers a dose greater than about 50 cGy/minute and less than about 300 cGy/minute.

9. An image-guided irradiation system according to claim 1, further comprising a radiation-shielding enclosure that at least partially encloses said support stage and said x-ray source.

10. An image-guided irradiation system according to claim 9, wherein said radiation-shielding enclosure is a portable radiation-shielding enclosure.

11. An image-guided irradiation system according to claim 1, further comprising a support frame to which said support arm is rotatably attached, to which said flat-panel x-ray detector is attached, and to which said stage-positioning assembly is attached, wherein said image-guide irradiation system is a portable image-guided irradiation system.

12. An image-guided irradiation system according to claim 11, further comprising a plurality of wheels attached to said support frame permitting said image-guided irradiation system to be rolled from a first position to a second position.

13. An image-guided irradiation system according to claim 1, further comprising an image processing system in communication with said flat-panel detector.

14. An image-guided irradiation system according to claim 13, further comprising an image display system in communication with said image processing system.

15. An image-guided irradiation system according to claim 13, further comprising a stage control system in communication with said support stage.

16. An image-guided irradiation system according to claim 13, further comprising a computerized irradiation planning system in communication with said image processing system, wherein said computerized irradiation planning system is adapted to receive information from said image processing system and to determine an irradiation plan based on said information from said image processing system and based on design information of the image-guided irradiation system.

17. An image-guided irradiation system according to claim 16, further comprising a stage control system in communication with said support stage.

18. An image-guided irradiation system according to claim 17, further comprising a computerized trajectory planning system in communication with said image processing system, wherein said computerized trajectory planning system is adapted to receive information from said computerized irradiation planning system and to determine a trajectory plan based on said information from said computerized irradiation planning system.

19. A portable image-guided irradiation system, comprising:
a portable imaging and irradiation system; and
a portable radiation-shielding enclosure that is suitable to substantially enclose said portable imaging and irradiation system.

20. A method of irradiating an object with X-rays, comprising:
illuminating an object with a first imaging beam of X-rays produced by an x-ray source from a first direction;
detecting X-rays from said first imaging beam of X-rays with a flat-panel x-ray detector after said illuminating to obtain first two-dimensional x-ray image data;
illuminating an object with a second imaging beam of X-rays produced by said x-ray source from a second direction;
detecting X-rays from said second imaging beam of X-rays with a flat-panel x-ray detector after said illuminating to obtain second two-dimensional x-ray image data;
processing said first and second two-dimensional x-ray image data to determine a three-dimensional x-ray image of said object;
and irradiating a selected portion of said object with an irradiation beam of X-rays produced by said x-ray source based on said three-dimensional x-ray image of said object,
wherein said irradiation beam of X-rays is narrower than said imaging beam of X-rays.

21. A method of irradiating an object with X-rays according to claim 20, further comprising at least one of collimating and focusing said irradiation beam of X-rays prior to said irradiating said selected portion of said object.

22. A method of irradiating an object with X-rays according to claim 20, wherein said irradiation beam of X-rays consist essentially of X-rays at higher energies than X-rays of said imaging beam of X-rays.

23. A method of irradiating an object with X-rays according to claim 20, wherein said irradiation beam of X-rays consists essentially of X-rays produced by an x-ray tube operated at a voltage greater than about 160 kVp and less than about 300 kVp, and
wherein said imaging beam of X-rays consists essentially of X-rays produced by said x-ray tube operated at a voltage greater than about 70 kVp and less than about 120 kVp.

24. A method of irradiating an object with X-rays according to claim 20, further comprising repeating said illuminating and said detecting a plurality of times at a plurality of different illumination directions prior to said processing to obtain refined information to obtain an improved three-dimensional x-ray image of said object.

25. A method of irradiating an object with X-rays according to claim 20, wherein said object is a laboratory test animal.

26. A method of irradiating an object with X-rays according to claim 25, wherein said laboratory test animal is a laboratory mouse.
